(19) United States
(12) Patent Application Publication
(10) Pub. No.: US 2024/0122492 A1
(43) Pub. Date: Apr. 18, 2024

(54) INTRAPLEURAL PRESSURE REGULATING DEVICE

(71) Applicant: Emre KARADENIZ, Kocaeli (TR)

(72) Inventor: Emre KARADENIZ, Kocaeli (TR)

(21) Appl. No.: 18/277,385

(22) PCT Filed: Dec. 22, 2021
(86) PCT No.: PCT/TR2021/051453

§ 371 Date: Aug. 16, 2023

(30) Foreign Application Priority Data
Feb. 17, 2021 (TR) 2021/002261

(51) Int. Cl. A61B 5/03; A61M 1/04

(52) U.S. Cl. CPC A61B 5/03; A61M 1/04; A61M 2205/3334; A61M 2205/3344; A61M 2210/101

(57) ABSTRACT

An intrapleural pressure regulating device that regulates the pressure in the intrapleural cavity and an operating method therefor are provided. The intrapleural pressure regulating device provides for removal of the foreign substances in the alveolar system and for supporting or replacing the functions of lungs and heart. With the intrapleural pressure regulating device, developed are a device regulating the pressure in the intrapleural cavity by pumping a fluid into the intrapleural cavity and withdrawing it from the intrapleural cavity, and an operating method for variable regulation of the pressure in the intrapleural cavity with said device.

### CROSS REFERENCE TO THE RELATED APPLICATIONS

This application is the national phase entry of International Application No. PCT/TR2021/051453, filed on Dec. 22, 2021, which is based upon and claims priority to Turkish Patent Application No. 2021/002261, filed on Feb. 17, 2021, the entire contents of which are incorporated herein by reference.

### TECHNICAL FIELD

The present invention relates to an intrapleural pressure regulating device that regulates the pressure in the intrapleural cavity and an operating method therefor.

### BACKGROUND

Fluids accumulating in the lungs due to diseases such as pneumonia or suffocation impair functioning of the lungs. In this case, there is a forced ventilation in the lungs.

Even in case of the impaired negative pressure in the intrapleural cavity due to a disease or trauma, in order for lungs to function properly, drainage is applied to this cavity to improve the patient's condition.

In case of cardiac and respiratory arrest, not only air is provided to the patient and but also blood is pumped by compressing the heart between the sternum and the spine with cardiopulmonary resuscitation (CPR) maneuvers.

Document US2002165618A1 discloses an inflatable implant used for lung support after lung reduction surgery. It also discloses the expansion and contraction of the implant with a pump in order to support the patient's breathing.

WO9855165A1 discloses an implant that applies regular pressure to the heart surface to support the heart.

Document U.S. Ser. No. 10/179,008B2 discloses a needle set for insufflation of the intrapleural cavity. It is mentioned that the needle can be opened directly to the atmosphere or connected to one or more fluid sources that allow a fluid to be pumped. It is also mentioned that this fluid can be connected to a vacuum source for withdrawing. It is also mentioned that the invention can be used to push the heart to the right during heart surgeries.

WO2006014567A2 discloses an assembly applying an adhesive to the intrapleural cavity, for the treatment of pleural effusion.

Document CA2704668A1 discloses a catheter that allows a fluid to be pumped into the intrapleural cavity and, in particular, the removal of fluids from the intrapleural cavity.

Document US2009240192A1 discloses an assembly for insufflation of body cavities, including the intrapleural cavity. It has been focused on delivering medication to the related cavities together with the gas pumped.

### SUMMARY

An object of the present invention is to develop a device that regulates the intrapleural pressure to act on the lungs.

Another object of the present invention is the development of a device that regulates the intrapleural cavity volume to act on the heart.

Yet another object of the present invention to develop an operating method for regulating the intrapleural pressure according to a periodic waveform with the device according to the invention, to support or replace the lung functions.

Yet another object of the present invention to develop an operating method for regulating the intrapleural cavity volume according to a periodic waveform with the device according to the invention, to support or replace the heart functions.

Yet another object of the present invention is to develop an operating method for regulating the intrapleural cavity volume so that the lung is deflated with the device according to the invention, to remove foreign substances in the alveolar system.

With the invention, a device has been developed that regulates the pressure in the intrapleural cavity by pumping and withdrawing a fluid to the intrapleural cavity and from the intrapleural cavity. This device basically comprises a delivery set delivering the fluid to the intrapleural cavity, at least one pump that can direct the fluid, and a control unit actuating the pump. A method has also been developed for operating the intrapleural pressure regulating device of the invention such that lung function and heart function are supported and replaced, if required, by variable regulation of the pressure in the intrapleural cavity.

### BRIEF DESCRIPTION OF THE DRAWINGS

The intrapleural pressure regulating device realized to achieve the objects of the present invention is shown in the attached figures.

FIG. 1 shows an intrapleural pressure regulating device according to the invention.

FIG. 2 shows a schematic view of an intrapleural pressure regulating device according to the invention.

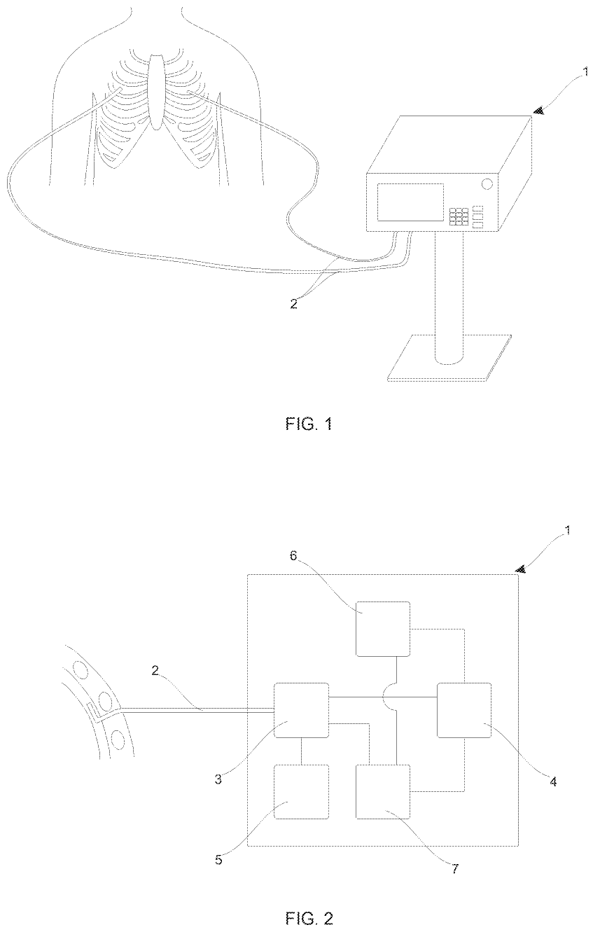

The components in the figures are numbered individually, and the equivalents thereof are given below.
1. Device
2. Cannula
3. Pump
4. Control unit
5. Internal reservoir
6. Controller interface
7. Power source

### DETAILED DESCRIPTION OF THE EMBODIMENTS

The intrapleural pressure regulating device (1) according to the invention, essentially, comprises the following:
a delivery set having at least one end suitable for placement into the intrapleural cavity and allowing bidirectional fluid passage,
at least one pump (3), to which the delivery set is connected and capable of pumping fluid in two directions,
a control unit (4) actuating the pump (3).

The delivery set comprises at least one cannula (2). Preferably, there is one cannula (2) for each of two lungs. A larger number of cannulas (2) can also be provided that reach different parts of the intrapleural cavity. The cannulas (2) may be suitable for bidirectional fluid passage, or there may be separate cannulas (2) for the fluid to be pumped into the intrapleural cavity and to be withdrawn from the intrapleural cavity.

The pump (3) or pumps (3) can be actuated by the control unit (4) in such a way as to withdraw the fluids contained in the intrapleural cavity into which the delivery set is reached, and to pump the fluid into the intrapleural cavity by means of the delivery set. Thus, the device (1), like its counterparts in the state of the art, can be used to bring the intrapleural pressure to a level that will support respiration by removing the fluids accumulated in the intrapleural cavity due to various reasons including trauma and inflammation, to deliver therapeutic substances such as medication to the intrapleural cavity, or to insufflate the intrapleural cavity.

Furthermore, the device (1) according to invention can also be used to support or replace the functions of lungs and heart. To that end, the pump (3) is actuated by the control unit (4) in a way to create variable intrapleural pressure.

The device (1) according to invention may be suitable for the use of gaseous or liquid fluids. Along with the fluid, solid, liquid and gaseous excipients, such as medication, carried by the fluid can also be delivered to the intrapleural cavity. Fluids can be supplied from an internal reservoir (5), an external reservoir or a line. Due to hygiene requirements, the internal reservoir (5) is replaceable. The internal reservoir (5) can be connected to the device (1) by filling with the desired fluid, or can be filled from the external reservoir or line after connecting to the device

The device (1) may comprise internal sensors measuring at least one variable associated with the flow rate of the fluid to allow detecting conditions that may prevent the desired intrapleural pressure regulation such as malfunctioning of the pump (3) or a blockage within the delivery set, and internal sensors measuring a variable associated with the fluid level within the internal reservoir (5). In addition, internal sensors can also be present that measure features such as density, light transmittance, and particle content of the fluid withdrawn from the intrapleural cavity to monitor the intrapleural cavity status.

The device (1) according to the invention, may also comprise one or more integrated external sensors and/or one or more connection points that allow data to be obtained from the external sensors, to measure findings related to the patient that can be used in order to determine the intrapleural pressure to be created or to evaluate the effectiveness of the attempt of regulating the intrapleural pressure. For example, external sensors such as a blood pressure monitor, a flow meter for evaluating respiratory efficiency, a balloon catheter for direct or indirect monitoring of intrapleural pressure can be used in combination with the device (1).

The device (1) also comprises a controller interface (6) that allows the control unit (4) to be operated by a healthcare personnel. The controller interface (6) comprises one or more of the components that allow to obtain inputs related to the intrapleural pressure to be regulated from the healthcare personnel and to provide outputs related to functioning of the device (1) to the healthcare personnel, such as one or more buttons, indicators, screen or touch screen, a wired or wireless connection device that allows access by a program executed on an external device. The outputs may include data obtained by or derived from the internal sensors and external sensors as well as information such as how the pump (3) is operated and the status of the internal reservoir (5).

The device (1) also comprises a power supply (7). The power supply (7) may consist of a battery embedded in the device (1), a connector connected to an external source, or a combination thereof.

The intrapleural pressure regulating device (1) will be operated in such a way as to regulate the intrapleural pressure in the form of a periodic or quasi-periodic waveform according to the frequency in order to support or replace the functions of lungs and/or heart with an intrapleural pressure regulating device (1) according to the invention. This waveform may consist of a combination of one or more functions. These functions may be combined piecewise or by superposition. The waveform may have fixed frequency and amplitude or variable frequency and/or amplitude. Different waveforms may also be used for two lungs. The waveforms used in this case are synchronized with each other.

In one embodiment of the invention wherein it is used to support or replace the lung functions, the waveform corresponds to the pressure change of the intrapleural cavity during the respiratory cycle. That is, the negative pressure required for adequate oxygenation is applied during inhalation, followed by alternating higher pressures required to remove carbon dioxide from the alveolar system during exhalation. Thus, the pressure difference between the intrapleural cavity and the alveolar system can be adjusted so that the respiratory cycle is performed.

In one embodiment of the invention wherein it is used to support or replace the heart functions, the waveform is such that it corresponds to the volume change of the heart during the cardiac cycle and that allows compression and relaxation of the heart in sequence for the thorax by changing the intrapleural cavity volume. In other words, following the application of intrapleural negative pressure during the relaxation of and expansion of the heart and filling of blood into the heart, positive pressure values are applied alternately to ensure the necessary volume contraction in the heart for pumping the blood in the heart into the arterial system. Thus, the volume of the intrapleural cavity can be adjusted to allow the cardiac cycle to be performed.

In one embodiment of the invention wherein it is used to support or replace both the lungs and heart functions, a wave function may be used that is obtained by combining portion wise a certain number of periods of a function corresponding to the respiratory cycle and a certain number of periods of a function corresponding to the cardiac cycle in sequence. In another embodiment, a wave function may also be used that is in the form of a superposition of a function corresponding to the respiratory cycle and a function corresponding to the cardiac cycle.

Characteristics of said waveform such as the shape, frequency and amplitude and the total application time, can be determined by the input provided by a healthcare personnel, or can be determined by arranging the values determined by the healthcare personnel according to data obtained from the external sensors. The data obtained from external sensors can determine whether the respiratory and cardiac cycles are performed by the patient spontaneously in embodiments for replacing lungs and/or heart functions as well as monitoring the effectiveness of the application, and can synchronize the waveform with the patient's spontaneous respiratory and cardiac cycle in embodiments for supporting lungs and/or heart functions.

An intrapleural pressure regulating device (1) according to the invention can also be used for removing the foreign substances in the alveolar system. To that end, the device (1) is operated in such that one or both lungs are deflated.

What is claimed is:

1. An operating method of an intrapleural pressure regulating device, wherein the intrapleural pressure regulating device comprises:
a delivery set having at least one end suitable for placement into an intrapleural cavity and allowing bidirectional fluid passage,
at least one pump wherein the delivery set is connected to the at least one pump and the at least one pump is configured to pump fluid in two directions, and
a control unit actuating the at least one pump;
wherein the operating method comprises: regulating an intrapleural pressure in a form of a periodic or quasi-periodic waveform according to a frequency and corresponding to a volume change of a heart during a cardiac cycle by changing an intrapleural cavity volume to compress and relax the heart in sequence.

2. The operating method of the intrapleural pressure regulating device according to claim 1, wherein the periodic or quasi-periodic waveform is a combination of a function corresponding to a pressure change of the intrapleural cavity during a respiratory cycle and a function corresponding to a pressure change of the intrapleural cavity during the cardiac cycle.

3. (canceled)

4. The operating method of the intrapleural pressure regulating device according to claim 1, wherein the periodic or quasi-periodic waveform consists of a combination of one or more functions.

5. The operating method of the intrapleural pressure regulating device according to claim 1, wherein the periodic or quasi-periodic waveform has variable frequency.

6. The operating method of the intrapleural pressure regulating device according to claim 1, wherein the periodic or quasi-periodic waveform has variable amplitude.

7. The operating method of the intrapleural pressure regulating device according to claim 1, wherein characteristics of the periodic or quasi-periodic waveform are determined by an input provided by a healthcare personnel.

8. The operating method of the intrapleural pressure regulating device according to claim 7, wherein values determined by the healthcare personnel are arranged according to data obtained from external sensors measuring findings related to a patient.

9. The operating method of the intrapleural pressure regulating device according to claim 2, wherein the periodic or quasi-periodic waveform is obtained by combining portion wise a certain number of periods of a function corresponding to the respiratory cycle and a certain number of periods of a function corresponding to the cardiac cycle in sequence.

10. The operating method of the intrapleural pressure regulating device according to claim 2, wherein the periodic or quasi-periodic waveform in a form of a superposition of a function corresponding to the respiratory cycle and a function corresponding to the cardiac cycle.

11. The operating method of the intrapleural pressure regulating device according to claim 1, wherein the intrapleural pressure regulating device is operated in such way that one or both lungs are deflated.
